(19) United States
(12) Patent Application Publication
(10) Pub. No.: US 2026/0091247 A1
(43) Pub. Date: Apr. 2, 2026

(54) AN ULTRASONIC DEVICE FOR SKIN TREATMENT

(71) Applicant: BORWARNRAT CAMPEERA, BANGKOK (TH)

(72) Inventor: BORWARNRAT CAMPEERA, BANGKOK (TH)

(21) Appl. No.: 19/110,162

(22) PCT Filed: Sep. 10, 2023
(86) PCT No.: PCT/TH2023/050027

§ 371 Date: Mar. 10, 2025

(30) Foreign Application Priority Data
Sep. 9, 2022 (TH) 2201005999

(51) Int. Cl. A61N 7/02; A61N 1/04; A61N 2/00; A61N 2/02; A61N 7/00; B06B 1/06

(52) U.S. Cl. CPC A61N 7/02; A61N 1/0452; A61N 2/002; A61N 2/004; A61N 2/02; B06B 1/0644; A61N 2007/0034; A61N 2007/0078

(57) ABSTRACT

The ultrasonic device for skin treatment, according to this invention, comprises of the external part consisting of the body of the device (2) where the middle of the body of the device (2) is installed with a light (3) for indicating the operation status of the device, the switch (4) for turning on and turning off the device and the mechanism parts which consists of the source of energy (6) that connects to the circuit control panel (7) contained with the short circuit detection circuit (8), the Bluetooth and/or Wi-Fi receiver and transmitter (9) and the skin temperature monitoring circuit (10). The circuit control panel (7) delivers the electric current to the electromagnetic electrodes (11), the electrical muscle stimulation electrodes (12) and the ultrasonic transducer (1) consisting of the deeper focal length ultrasonic transducer (17) and the shallower focal length ultrasonic transducer (13). Both of the transducers consist of the electrodes connecting to the piezo electric material for delivering the electricity to the piezoelectric material and the solid or semi-solid material such as polymer, metal or gel, which is ultrasonic transmittable for intensifying the ultrasonic wave into the desired skin layer. There is the temperature monitoring probe (21) near the ultrasonic transducer (1) which is connecting to the temperature monitoring circuit (8).

### TECHNICAL FIELD

Biomedical and electrical engineering is related to the ultrasonic wave for skin treatment.

### BACKGROUND ART

Nowadays, the non-invasive skin lifting management is getting more popular. The principle is stimulating the skin underneath which will then lead to the neocollagenesis and neofibrogenesis. The prior technology is phonophoresis which generates low power ultrasonic wave by transforming electricity into low powered ultrasonic wave by piezoelectric material. Another technology is iontophoresis which releases very low electric current directly into the skin. Both technologies are unsatisfactory and the result is not explicit because the concentration of ultrasonic wave is low and clustered only in superficial layer of the skin in the area where the device touches the skin. Therefore, it is unable to generate heat into the deeper skin in the desired depth for neocollagenesis and neofibrogenesis. Like phonophoresis, iontophoresis's electric current released is very low, and concentrates only around the electrodes which is unable to generate heat into the deeper skin in the desired depth for neocollagenesis and neofibrogenesis.

After that, there is an updated technology called HIFU (High Intensity Focused Ultrasound) which is then applied in dermatology for non-surgical skin laxity management. This technology transforms electricity into ultrasound wave, then the ultrasound wave is emitted and compressed in dotted (Dotted High Intensity Focused Ultrasound: Dotted HIFU) wave causing higher intensity of the ultrasound. The intensity depends on the energy supplied and the design of the curved piezoelectric material. Basic researches state that the focal point energy can reach up to 1-3 folds compared to the conventional device which is emitted without intensifying. Based on this principle, the emitted ultrasound wave generates the heat at the deeper skin layer which the focal depth can be designated. This focal depth is the area where the intensified ultrasonic wave is highest condensed because of the curved piezoelectric material. In case the focal point is desired to be deeper, the radius of curvature will be long. In case the focal point is desired to be shallower, the radius of curvature will be short. In this regard, the temperature of skin at the focal point can be reached up to 60-70 degree Celsius which is the best temperature for protein denaturation leading to the inflammation and wound healing process which are the main processes for the neocollagenesis and neofibrogenesis. However, there is still some limitation on such the mentioned device; that is the focused ultrasonic wave transmits the energy dot by dot in dotted line form moving the ultrasonic transducer along the assigned axis powered by a motor which takes a long time for the treatment. Furthermore, the high energy at focal point causes extreme pain while and after using the device. This principle is applied by the device called Ultherapy (Ultherapy®: US20200100762A1) for skin lifting which, although such device provides a very good results in solving the problems and it is done only once a year, causes an extreme pain due to such a high energy emitted and time consuming.

Based on the search results, the Dotted HIFU under the brand named Ultracel Q+ produced by Jeisys company is launched with the new technology. The ultrasound transducer moves along the assigned axis and powered by a motor and emits ultrasonic wave into a dotted form but continually emits this dotted ultrasonic wave which results in a linear focal energy. However, there is still no other device which transmits a true straight line focal energy and no simultaneously transmits greater than or equal to 1 frequency and 2 focal depths. If the user needs to switch the frequency or the desired focal depth, the user needs to swap the transducer. Furthermore, most of the previous devices use liquid, such as water, as an ultrasonic wave medium which must spare an air in the transducer chamber because, when the water is heated from the transmitting ultrasonic wave, the water is boiled and evaporated. In addition, there must be a membrane to prevent a water leakage from the transducer chamber which all these conditions are inconvenient and hugely affecting to the operation of the device and the results of treatment, especially when the device is overturned or upturned. When the transducer is upturned, the air in the transducer chamber floats below the membrane which causes wrong ultrasonic wave refraction leading to the change of focal length, then changes of the energy at the focal point causing adverse effect from the use of device, i.e., skin burn. Therefore, this new device is invented under the concept of transmitting the ultrasonic wave where the energy at the focal point is reduced resulting in the reduction of the pain as well as solving the problems as described above.

### NATURE AND PURPOSES OF THE INVENTION

The ultrasonic device for skin treatment, according to this invention, consists of the external part which the body of the device (2) is in a form of cylindrical square bar where its end contains mechanism parts. The middle of the body of the device (2) is installed a light (3) for indicating the operation status of the device and the switch (4) for turning on and turning off the device. The mechanism part consists of the energy source (6) which connects to the circuit control panel (7) containing the short circuit detection circuit (8). The circuit control panel (7) adjusts the electric current and transmits the electric current to the electromagnetic electrodes (11), the electrical muscle stimulation electrodes (12) and the ultrasonic transducer (1) which consists of the deeper focal length ultrasonic transducer (17) and the shallower focal length ultrasonic transducer (13). Both of the transducers generate and intensify ultrasonic wave then transmit in a form of the small straight cylindrical shape wave. The ultrasonic transducer (1) consists of the electrodes (14, 18) which deliver the electricity to the piezoelectric material (15, 19). The electrodes (14, 18) connect to the concave side of the piezoelectric material (15, 19) which converts the electric current into the ultrasonic wave, while the concave side of the piezoelectric material (15, 19) connects to the solid or semi-solid material (16, 20) such as polymer, metal or gel which is transmittable for ultrasonic wave. The solid or semi-solid material (16, 20), cubic shape, transmits focuses and intensifies the ultrasonic wave into the desired depth of the skin. There is no motor for moving the ultrasonic transducer (1) and the ultrasonic transducer (1) is fixed in end of the body of the of the device (2). Near the ultrasonic transducer (2) there is the temperature monitoring probe (21) which connects to the temperature monitoring circuit (8) installed in the circuit control panel (7).

The purpose of this invention is the ultrasonic device for skin treatment which is able to simultaneously transmit at least 1 frequency and 2 focal points, shallower focal length and deeper focal length, for targeting the main skin layers;
1. SMAS (superficial Musculoaponeurotic System), the fascia layer of muscle which is located in the deeper skin layer.
2. Dermis which is located in the shallower skin layer.

This stimulates neocollagenesis and neofibrogenesis in all essential skin layers. In addition, the device is able to transmits electromagnetic wave for the extended indications of skin problems management such as excessive subcutaneous fat management and synchronously stimulates neocollagenesis and neofibrogenesis for skin laxity management together with the focused ultrasonic wave. Furthermore, the device is able to emits the low electric current for increasing muscles toning. All these technologies will be integrated in one device to solve skin problems in the same time. The prior technology is solely 1 frequency focal point, causes an extreme pain, and wastes time. For the different skin depth treatment, the user must change the transducer which is inconvenient and the device must be operated by the expert only because of the very high energy. Non-medical persons cannot use such the device. Besides, this new device changes the ultrasonic wave medium from liquid to solid or semisolid material, cubic shape, such as polymer, metal or gel which are able to constantly transmit ultrasonic wave and the position of the transducer does not affect the refraction of the ultrasonic wave, focal point length and the energy. This problem-solving technology let the user conveniently freely move the device on the skin without effecting the ultrasonic wave refraction. Compared to the previous technology which uses the liquid for the ultrasonic wave medium. The liquid boils and evaporates when the ultrasonic wave transmit through. Because of this reason, the transducer chamber must leave an air gap inside otherwise the transducer may explode. Furthermore, the transducer must place a film for protecting the water leakage. When the transducer is upturned, the air floats upwardly and locates between the film and the water which affects the wave refraction including focal point energy and length.

In conclusion, this new device is invented for solving previous technology which is a single frequency and single focal point, intensifies ultrasonic wave into dot and uses liquid as an ultrasonic wave medium. The previous technology transducer moves along the assigned axis powered by the motor creating a dotted line ultrasonic wave but this his new technology spreads the ultrasonic wave into a true small straight line which covers a larger area and reduces treatment time. The position of the transducer does not affect the operation of the device with stably transmitted intensified ultrasonic wave. The device can transmit simultaneously at least 1 frequency and 2 focal points without changing the ultrasonic transducer. The operator can be anybody, not only medical professionals. The most important concept is causing less or no pain whether during or after using this device.

### BRIEF DESCRIPTION OF THE DRAWINGS

FIG. 1 is the external part.

FIG. 2 is the mechanism part and the connection.

FIG. 3 is the ultrasonic focal point.

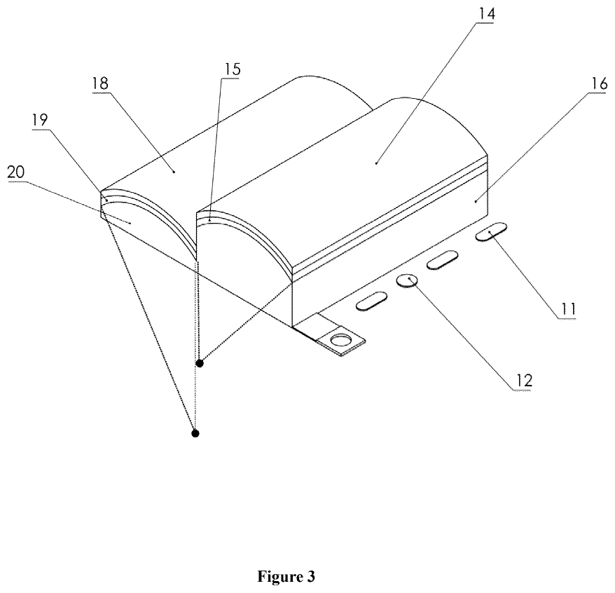

FIG. 4 is the cross section of the internal mechanism part.

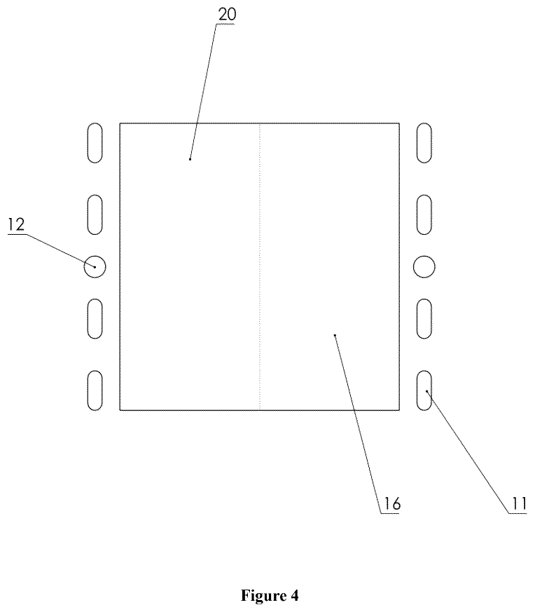

### DISCLOSURE OF THE INVENTION

The ultrasonic device for skin treatment consists of:

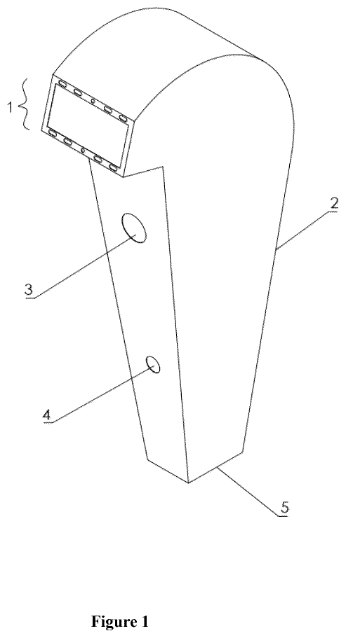

FIG. 1 is the external part which consists of the body of the device (2) in a form of cylindrical square bar where there is the mechanism part in its end for generating the focused ultrasonic wave and transmitting the focused ultrasonic wave to the desired skin layer. The middle of the body of the device (2) is installed a light (3) for indicating the operation status of the device and the switch (4) for turning on and turning off the device. In the other end of the body of the device (2), there is an opening channel (5) for connecting to the energy source (6).

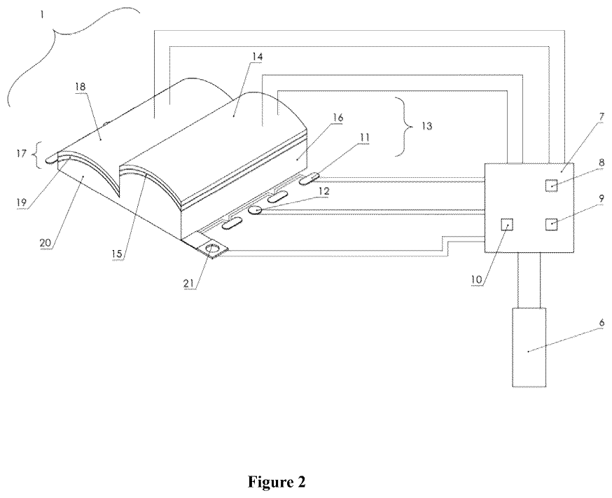

FIG. 2 is the mechanism part and the connection consists of the energy source (6) for supplying the electricity to all the device systems which connects to the circuit control panel (7). The circuit control panel (7) regulates electricity, cuts electricity, turns on and turns off the device by the connected switch (4). The circuit control panel (7) consists of the short circuit detection circuit (8), if there is a short circuit the electricity will be cut and the operation of the device will be paused. (7) The circuit control panel (7) additionally contains the Bluetooth and/or Wi-Fi receiver and transmitter (9) for receiving and transmitting the operating data of the device and connecting with other devices. In addition, the circuit control panel (7) connects electromagnetic electrodes (11), electrical muscle stimulation electrodes (12) and the ultrasonic transducers (1) consisting of the shallow focal length ultrasonic transducer (13) and the deep focal length ultrasonic transducer (17). Each of the ultrasonic transducer consists of the piezoelectric material (15, 9) which is curved. The convex side of the piezoelectric material (15,19) is fixed with both anode and cathode electrodes for transmitting the electric current to the piezoelectric material (15, 19) while the concave side is fixed with the solid or semi-solid material (16, 20) such as polymer, metal or gel which is transmittable for ultrasonic wave in a form of solid or semi-solid cube. The upper surface of the solid or semisolid material (16, 20) is curved which is fixed to the concave side of the curved piezoelectric material (15,19). The opposite side is flat or slightly curved. The transducers (1) are fixed and immobilized in the end of the body of the device (2)

Next to the ultrasonic transducer (1), there is a temperature monitoring probe (21) for measuring epidermal temperature while operating the device. If the temperature is beyond the setting temperature, the temperature monitoring circuit (10) will cut the electricity to the ultrasonic transducer (1). If there is a short circuit, the short circuit detection circuit (8) will totally cut the electricity in the device for safety. Next to the ultrasonic transducer (1), there is the electromagnetic wave electrodes (11) which transmits electromagnetic wave for the excess subcutaneous fat management and synergistically with the focused ultrasonic wave stimulates neofibrogenesis and neocollagenesis and electrical muscle stimulation electrodes (12) which transmits low electric current to the muscles for strengthening and toning up muscles.

The deep focal length ultrasonic transducer (17) generates and intensifies straight columnar 0.1-20 megahertz (MHz) and 2.5-30 millimeter (mm.) ultrasonic wave mainly targeting SMAS layer (deeper layer). The deep focal length ultrasonic transducer (17) consists of the curved piezoelectric material (19) which the concave side is fixed to the electrodes (18) for delivering the electricity from the circuit control panel (7) to the piezoelectric material (19). After that, the piezoelectric material (19) will convert electricity into the ultrasonic wave. The concave side of the piezoelectric material (19) is fixed to the solid or semi-solid material (20) such as polymer, metal, or gel. The solid or simi-solid material (20) is cubic shape which is ultrasonic wave transmittable where the upper side is curved and the opposite side is flat or slightly curved. The curved side of the solid or semi-solid material (20) is connected to the piezoelectric material (19) where its radius of curvature is consistent with the radius of curvature of the piezoelectric material (19) for conducting and intensifying the ultrasonic wave into a smaller wave beam transmitting to the desired depth of skin layer.

The shallow focal length ultrasonic transducer (13), the shallower focal length, generates ultrasonic wave and intensifies into a straight columnar 1-25 MHz and 1-25 mm. ultrasonic wave mainly targeting the dermis layer (shallower layer). The shallow focal length ultrasonic transducer consists of the piezoelectric material (15) which the concave side is fixed to the electrodes (14) for delivering the electricity from the circuit control panel (7) to the piezoelectric material (15). After that, the piezoelectric material (15) will convert electricity into the ultrasonic wave. The concave side of the piezoelectric material (15) is fixed to solid or semisolid material (16) such as polymer, metal, or gel. The solid or simi-solid material is cubic shape which is ultrasonic wave transmittable where the upper side is curved and the opposite side is flat or slightly curved. The curved side of the solid or semi-solid material (16) is connected to the piezoelectric material (15) where its radius of curvature is consistent with the curvature of the piezoelectric material (15) for conducting and intensifying the ultrasonic wave converted from the electricity and intensifying the ultrasonic wave into a smaller wave beam transmitting to the desired depth of skin layer.

The operation of the ultrasonic device for the skin treatment starts with receiving the signal from the switch (4) then the light (3) emits for indicating the device operation status. The circuit control panel (7) consists of the skin temperature monitoring circuit (10) which receives the temperature data from the temperature monitoring probe (21) located near the ultrasonic transducer (1) for measuring epidermal temperature. If the temperature exceeds the setting, the the temperature monitoring circuit (10) on the circuit control panel (7) will cut the electricity to the ultrasonic transducer (1). Near the ultrasonic transducer (1) there is also the electromagnetic wave electrodes (11) and the electrical muscle stimulation electrodes (12). This ultrasonic device for the skin treatment is able to add the additional ultrasonic transducer (13,17) at least 1 frequency and at least 2 focal lengths for extending the indication of the treatment by joining transducers together.

The advantage of this newly invented ultrasonic transducer is transmitting the truly straight columnar intensified ultrasonic wave along the length of the solid or semisolid material (20, 16) such as polymer, metal, or gel, which is ultrasonic transmittable. The energy of the ultrasonic wave is evenly spreading, not dotted focusing like prior technology but still intensifies ultrasonic wave which is able to raise skin temperature to 40-55 degree Celsius. This temperature level, sub optimal temperature for neocollagenesis and neofibrogenesis, still is able to stimulate new collagen and fibroblast synthesis but not as much as 60-70 degree Celsius. The heat transmitted through the epidermis is low which is good for skin burn prevention but still intensifies the ultrasonic wave in the skin layer below the epidermis. This device can treat multiple skin problems at the same time, for example, skin laxity, excessive subcutaneous fat or poor muscle toning. The prior technology, to solve these problems, needs several devices. Therefore, the inventor creates the device to solve multiple problems in only one device.

### BEST MODE FOR CARRYING OUT THE INVENTION

Same as disclosed in the part of Disclosure of the Invention.

1. The ultrasonic device for skin treatment consisting of the two main parts; external part and mechanism part
The external part of the body of the device (2) where its end consists of the mechanism part which generates and delivers ultrasonic wave to the skin. The middle of the body of the device (2) consists of switch (4) for turning on and turning off the device. Another end of the body of the device (2) consists of the part for connecting to the energy source (6).
The mechanism part is located in the external part, consisting of energy source (6) for delivering electric current to the whole systems of the device which connects to the circuit control panel (7) for turning-on and turning-off by the connected switch (4)
Where the special feature is the circuit control panel (7) connecting to the source of energy (6) and delivering the electric current to the ultrasonic transducer (1) which consists of the shallower focal length ultrasonic transducer (13) and the deeper focal length ultrasonic transducer (17). The ultrasonic transducer (1) is fixed and immobilized in the end of the body of the device (2).
The shallower focal length ultrasonic transducer (13) consists of the electrodes (14) for delivering the electric current from the circuit control panel (7) which is fixed to the curved piezoelectric material (15) at the convex side. The concave side of the piezoelectric material (15) is attached to the solid or semi-solid material (16), cubic shape, which is transmittable for the ultrasonic wave. The curvature radius of the upper side of the solid or semi-solid material (16) is consistent with the radius of the concave side of the piezoelectric material (15) where another side thereof is flat or slightly curved.
The deeper focal length ultrasonic transducer (17) consists of the electrodes (18) for delivering electric current from the circuit control panel (7) which is fixed to the curved piezoelectric material (19) at the convex side. The concave side of the piezoelectric material

(19) is attached to the solid or semi-solid material (20), cubic shape, which is transmittable for the ultrasonic wave. The curvature radius of the upper side of the solid or semi-solid material (20) is consistent with the radius of the concave side of the piezoelectric material (19) where another side thereof is flat or slightly curved.

2. The ultrasonic device for skin treatment according to claim 1 wherein the number of the ultrasonic transducer can be greater than or equal 2 at least 1 frequency and 2 focal lengths.

3. The ultrasonic device for skin treatment according to claim 1 or 2 wherein the shallower focal length ultrasonic transducer (13) generates 1-30 MHz and 1-25 mm. focal length ultrasonic wave.

4. The ultrasonic device for skin treatment according to any of claims 1-3 wherein the deeper focal length ultrasonic transducer (17) generates 0.1-20 MHz and 2.5-120 mm. focal length ultrasonic wave.

5. The ultrasonic device for skin treatment according to any of claims 1-4 wherein there is a temperature monitoring probe (21) which locates at the side of the ultrasonic transducer (1) for sensing the temperature signal to the temperature monitoring circuit (10).

6. The ultrasonic device for skin treatment according to any of claims 1-5 wherein there is the electromagnetic electrodes (11) which locates at the side of the ultrasonic transducer (1).

7. The ultrasonic device for skin treatment according to any of claims 1-6 wherein there is the electrical muscle stimulation electrodes (12) which locates at the side of the ultrasonic transducer (1).

8. The ultrasonic device for skin treatment according to any of claims 1-7 wherein the circuit control panel (7) can be additionally installed the short circuit detection circuit (8).

9. The ultrasonic device for skin treatment according to any of claims 1-8 wherein there is Wifi and/or Bluetooth receiver and transmitter installed in the circuit control panel (7).

10. The ultrasonic device for skin treatment according to any of claims 1-9 wherein there is a light (3) for indicating the operation status of device installed in the middle of the body of the device (2).
